(19) United States
(12) Patent Application Publication
(10) Pub. No.: US 2022/0068489 A1
(43) Pub. Date: Mar. 3, 2022

(54) COMPUTER MODELING AND EVALUATION OF INSURANCE PRICING AND RISK

(71) Applicant: CERNER INNOVATION, INC., KANSAS CITY, KS (US)

(72) Inventors: Douglas S. McNair, Leawood, KS (US); Kanakasabha Kailasam, Olathe, KS (US); Daniel Martin Feimster, Shawnee, KS (US)

(21) Appl. No.: 17/368,524

(22) Filed: Jul. 6, 2021

Related U.S. Application Data
(63) Continuation of application No. 14/982,978, filed on Dec. 29, 2015, now abandoned.
Application No. 14/982,978 is a division of application No. 13/758,494, filed on Feb. 4, 2013, now abandoned.
(60) Provisional application No. 61/594,766, filed on Feb. 3, 2012.

(51) Int. Cl. G16H 50/30

(52) U.S. Cl. CPC G16H 50/30

(57) ABSTRACT

Systems, methods and computer-readable media are provided for determining a premium for a proposed insurance coverage. In various embodiments, a plurality of context variables can be defined related to proposed coverage. Based on defined context variables, historical claim events are extracted from one or more sources of claims data. Extracted historical claim events may be representative of the proposed coverage. Historical claim events can be aggregated based on a time period to form aggregated claims data per time period. The aggregated data can then be used to determine a historical volatility time series based on differences between logarithms of the aggregated claims data per time period for a plurality of time periods. Additionally or alternately, the aggregated claims data per time period can be used to determine a fit of the aggregated data to one or more distributions. The fit can be evaluated, such as based on a fit parameter, to select a distribution. A calculation can then be performed to determine a premium. If a normal distribution is selected, the historical volatility time series can be used to estimate a volatility. This estimated volatility can then be used to calculate a premium.

### CROSS-REFERENCE TO RELATED APPLICATIONS

This application is a Continuation of U.S. patent application Ser. No. 14/982,978 filed Dec. 29, 2015, entitled “Computer Modeling and Evaluation of Insurance Pricing and Risk,” which is a divisional of U.S. patent application Ser. No. 13/758,494, filed Feb. 4, 2013, entitled “Computer Modeling and Evaluation of Insurance Pricing and Risk,” which claims priority to U.S. Provisional Application No. 61/594,766, entitled “Computer Modeling And Evaluation Of Insurance Pricing And Risk,” filed Feb. 3, 2012. The aforementioned applications are hereby incorporated by reference herein in their entirety.

### INTRODUCTION

One of the main risks facing insuring firms and individual insurance consumers alike arises when the cost per insured individual or insured group becomes excessive. Therefore, it is desirable to achieve an efficient insurance market where insurance premia are priced in an objective and sustainable manner and where prices accurately and transparently reflect the risks experienced by both parties to the contract.

An increasing number of smaller groups are self-insuring in order to reduce costs. Commercial insurers are developing products along similar lines to serve smaller pools, sometimes comprised of 50 or fewer insureds. While these insurance products corresponding to smaller pools offer some benefits, such plans also have high variability. In particular, a single high cost insurance event can potentially overwhelm the resources of a limited size plan. One solution for smaller insurance pools is to purchase some type of reinsurance, such as excess loss insurance or Medical Stop-Loss. Reinsurance is a mechanism that protects an insurer against the risk of covering insureds with high-cost financial exposures, such as diseases that require expensive treatments.

### SUMMARY

Systems, methods and computer-readable media are provided for determining a premium for a proposed insurance coverage. In various embodiments, a plurality of context variables can be defined related to the proposed coverage. Based on the defined context variables, historical claim events can be extracted from one or more sources of claims data. Preferably, the extracted historical claim events are representative of the proposed coverage. The historical claim events can be aggregated based on a time period to form aggregated claims data per time period. The aggregated data can then be used to determine a historical volatility time series based on differences between logarithms of the aggregated claims data per time period for a plurality of time periods. Additionally or alternately, the aggregated claims data per time period can be used to determine a fit of the aggregated data to one or more distributions. The fit can be evaluated, such as based on a fit parameter, to select a distribution. A calculation can then be performed to determine a premium. If a normal distribution is selected, the historical volatility time series can be used to estimate a volatility. This estimated volatility can then be used to calculate a premium using a Black-Scholes model. Selection of a different distribution can lead to other types of methods for determining a premium, such as using a jump-diffusion model or Monte Carlo sampling of a selected distribution.

### BRIEF DESCRIPTION OF THE DRAWINGS

The present invention is described in detail below with reference to the attached drawing figures, wherein:

FIG. 1A depicts aspects of an illustrative operating environment suitable for practicing embodiments of the invention;

FIG. 1B depicts an exemplary framework of a multi-agent system suitable for implementing an embodiment of the invention;

FIG. 2 shows an example of a volatility time series suitable for use in an embodiment of the invention;

FIG. 3 shows an example of a distribution representing a distribution of claim values according to an embodiment of the invention;

FIG. 4 shows an example of a volatility time series suitable for use in an embodiment of the invention; and

FIGS. 5-7 show examples of process flows according to an embodiment of the invention.

### DETAILED DESCRIPTION

### Overview

In various embodiments, systems and methods are provided that enable data mining, data processing, and/or modeling of a distribution of claim values to determine valuations associated with various types of insurance. The systems and methods allow for evaluation of the risk and reward associated with offering an insurance product at a particular premium level. This evaluation is enabled in part by the use of one or more autonomous computer agents that assist with the data mining, data processing, and simulation tasks.

For example, a system and method can be provided for calculating a minimum premium for an insurance product tailored based on a group of potentially insured individuals, a set of covered conditions, one or more coverage thresholds, or a combination thereof. A plurality of option calculation models are formulated wherein each of the plurality of models is based on established methods for calculating call options, such as call options for stocks or other equities. The method also includes the steps of (a) selecting a statistical prediction limit (alpha), the risk-free interest rate, the cost-of-carry rate, and the term of the option (the period during which the insurance policy shall be in-force); (b) determining the daily volatility from the log-returns time series of historical payouts for the target insureds or for a cohort of insureds that closely resembles the target insureds, and employing this volatility time series to forecast the volatility that likely will prevail during the term of the proposed insurance policy; (c) calculating a value of at least one European-style call option by Black-Scholes, jump-diffusion, Monte Carlo, or other methods, and (d) determining which among the call values best reflects the requirements of the insurer, so as to adequately protect the solvency of the insurer

Determining whether a premium is sufficient to cover the risk of an insurance product is an ongoing problem. Actuarial methods can provide some insight into the required premium. However, in situations where the group of insureds is small and/or where the amount of available historical data is limited, actuarial methods can have large uncertainties. As a result, some insurance premiums determined by conventional methods are priced at excessive levels in order to account for the uncertainty. Part of the reason for setting excessive premiums is that conventional actuarial methods struggle to correctly account for low frequency but high value claims. The impact of these claims on coverage is typically underestimated by conventional methods, which can lead to situations where the premiums paid are insufficient to cover liabilities.

An improved method of determining premium prices for insurance would allow an insurer to expand the potential pool of people or groups that can be insured while improving identification of the price required to appropriately mitigate the risk to the insurer. Ideally, such an improved method would also be suitable for use on a time scale of seconds minutes rather than hours or days. This would allow one or more aspects of a proposed coverage plan to be varied to investigate the cost of alternative coverage options, so as to provide alternatives for a potential insured group on a time scale that is responsive to customer needs. In various embodiments, this is achieved by using one or more autonomous agents to perform data mining, data processing, and calculations or simulations to determine an appropriate premium price.

In various embodiments, the insured/insurer relationship is modeled using an analogy to options contracts for the purchase and sale of equities. In this model, the insureds are analogous to investors who have primary positions in a particular equities sector, such as options, futures, or other equities derivatives, and who have secondary positions in other securities. Individuals' claims are analogous to expiries (liquidations) of European style call options. A European style call option refers to a call that is exercised at the end of an option period, as opposed to at some other time. The sum of the insurance premium plus the deductible is like the options' strike price (the individual has the right to buy the cumulative treatments at a price equal to the deductible plus the premium). Under this type of model, an insurer is profitable when the strike price of the “option” equals or exceeds the extreme-value distributed payout, which is the cumulative annual cost of treatment less the deductible. If this criteria is not satisfied, the insurance plan has an increased risk of becoming insolvent.

In this discussion, reference will be made to insurance coverage or insurance products. Unless otherwise specified, it is understood that the various embodiments described herein are also applicable to reinsurance coverage or reinsurance products, as well as other analogous types of products.

The systems and methods described herein can be useful for generating coverage premiums for a variety of types of insurance or reinsurance. One potential application is for hospitals or other facilities that provide care to patients. A patient receiving care may have an insurance policy that covers care up to a limit, such as one million dollars. When a patient exceeds this limit, the patient may lack the ability to pay for the difference, resulting in the hospital having a patient whose care must be continued without compensation. While the hospital can typically absorb these costs, an unusual year could result in a hospital receiving more of these patients than normal, resulting in large losses for the hospital. To avoid this risk, a hospital can buy insurance to cover this situation.

In another example, an employer may have a sufficient number of employees that the company decides to self-insure. However, the company would like to avoid the risk of having runaway costs due to a few unusual events. The company can buy reinsurance so that the risk of high value claims is transferred to another party.

Initial Construction of Model—Comparison with Options Contracts

A model for determining an insurance premium price for a tailored insurance product can be compared with a model for the structure of an options contract for the purchase of an equity instrument. This comparison is not essential for applying the model to develop a premium for a proposed coverage. Instead, the following discussion is provided merely to facilitate understanding of the model.

The profit-loss pattern associated with insurance operations can be replicated using option contracts. Both can be viewed as hedging operations. For example, an option covers the option-holder against unexpected changes in prices, while insurance covers insureds against unexpected contingencies, such as accidents or illness. Additionally, a premium is paid in both types of contracts. The option buyer or insured person pays a premium to the option writer or insurer in order to obtain the desired hedge or insurance. Another similarity is that when a triggering event occurs, compensation is paid. If, for example, an episode of illness occurs, the compensation consists in indemnifying the insured against the market price of treatments that are above the amount of the policy's deductible amount. This is analogous to an option strike price. If the market cost of the cumulative treatments received exceeds the deductible, the buyer implicitly executes the option and receives an amount (or is shielded from being billed for an amount) equivalent to the difference between the strike price and the market price of the services. Still another similarity is that the timing of both insurance and option operations is relatively short. Despite the fact that the time horizon of insurance coverage can be set in years, it is more typical for pricing to be set on a yearly basis. Similarly, options contracts may be established for short or long periods, but options are seldom contracted in current financial markets for periods longer than a year.

Based on the above, the premiums and payouts for an insurance (or reinsurance) operation have some similarity to pricing and payouts for option contracts. Based on this comparison, a securities type model for pricing options contracts can be useful in determining insurance premiums. Examples of suitable models include the Black-Scholes model, the jump-diffusion model, a model used in conjunction with Monte Carlo simulations to sample a model distribution, or other option pricing models suitable for calculating the fair-market value of a premium to be paid for specified insurance (or reinsurance) coverage.

### Mining of Historical Claims Data

As an initial step in evaluating pricing of an insurance product based on an options-style model, a set of historical data can be identified. This historical data set can be mined and processed to provide information for describing the future claims behavior of the proposed insurance product. The identified historical data can be selected so that the data is representative of the proposed type of insurance for the proposed group of insured subjects. The data can be selected to be representative based on a type of condition, a monetary constraint, or any other convenient feature. The data can be based on a single insured group or on an aggregation of various insured groups.

For example, one type of insurance coverage product is insurance that is directed to specific types of conditions, such as conditions related to heart disease. A potential group of insureds may desire additional coverage specifically for medical treatments related to heart disease. To value this type of insurance, historical data can be mined to extract only claim events related to heart conditions that would be covered by the insurance product.

Another filter for historical claims data is to select data based on monetary thresholds for a proposed insurance product. For example, an insurance product may have a minimum value before any claims are paid, such as a deductible. Additionally, the insurance product may have a maximum value beyond which no further claims are paid, such as a one year or multi-year cap on payments. When a new insurance product is proposed, such minimum and maximum payment values or thresholds can be specified, along with any other desired thresholds. The various thresholds can then be used to select claim activity from the historical data that includes only claims that would result in payment under the proposed terms of the insurance product.

The historical claims data can be extracted from any convenient data source. Examples of potential claims data include billing records sent to patients, claims submitted to insurers, or possibly even internal records from a health care provider of claims or bills that were not sent out due to inability to identify a potential payment provider. Examples of entities that could provide claims data include hospitals, walk-in clinics, and other health care providers. The claims data can be retrieved directly from an entity that submits the bill or claim or from a centralized medical record store, such as a store of medical records provided by a health information exchange.

Because of the wide variety of potential sources of historical claims data, a system that can launch a plurality of autonomous agents can be helpful in extracting the data. Depending on the proposed coverage, millions of claims may need to be filtered in order to identify the appropriate claims that match the defined coverage parameters. This task can preferably be performed in parallel by multiple agents.

Other processing can also be performed on the data prior to use. For example, inflation is an ongoing phenomenon in most economies, so that the dollar value of claims from one year needs be “inflation adjusted” in order to provide an accurate comparison between multiple years. In an embodiment, historical claims data can be adjusted so that all data points within a data set are valued on a consistent basis, such as valuing all claims in money (such as dollars) from the current year.

After selecting the appropriate historical data, the historical data can be organized as a time series of data or in another manner that is convenient for a subsequent simulation based on the data. For example, the daily mean value of the medical claims for all the days in the calculation period can be determined. The medical claims can represent claim amounts submitted to an insurer, amounts billed to a client, or another amount that corresponds to the cost of treatment. In some situations, the claim amount will not correspond to a payment received by a care provider, as a patient may lack the ability to pay. This is not relevant to the model, however, as the goal is to determine what the medical claims payout would be for a solvent insurer. Note also that the historical medical claims data can be filtered to exclude any claims that fall below a minimum or above a maximum value for the insurance product. Based on this information, the accumulated value of daily claims over the previous 12 months can be determined for each day in the historical time period. This provides a daily value for the net payout that would have been required for an insurance product purchased one year earlier that covered the members of the group corresponding to the historical data. If an insurance product for a six month period (or another convenient time period) is desired, that time period can be used instead.

Preferably, the historical data set can include historical claims for a sufficient period of time to allow for some confidence in the eventual results. For example, having at least 24 months of historical data can be helpful, although shorter periods of historical data may also be suitable for use.

In an equity asset value setting, it is believed that the values for an underlying asset during a given time period have a relationship with a normal or Gaussian distribution. This relationship is believed to be a lognormal relationship. In other words, plotting the logarithm of the value of an equity asset at each sampling point during a time period should give you a frequency distribution of log values that corresponds to a normal or Gaussian distribution. Any convenient base can be used for the log conversion, such as using a base 10 logarithm or a natural logarithm. This same assumption will be used for embodiments where a Black-Scholes calculation is performed. In order to take advantage of this assumption, a frequency plot does not need to be made. Instead, the values needed for a Black-Scholes calculation can be derived by building a time-series of the data using the log values of the claims.

After calculating a time series of the log values of the claims, a daily volatility can also be determined. As mentioned above, various embodiments of this invention are related to calculating or modeling insurance premiums based on an analogy with options pricing. For an equity underlying an option contract, the price of the underlying equity on a given day has a dependence on the value of the equity on the prior day. In this type of situation, a volatility between consecutive days (or another consecutive time unit) can be constructed. For example, using a time series based on the natural log of claims, the volatility between day 1 and day 2 can be expressed as ln(day 2)−ln(day 1). Because the volatility is what is desired, a positive or negative change is treated the same. If desired, this can also be expressed as ln(day 2/day 1).

Based on the volatility time series, a predicted or forecast volatility can be determined. The predicted or forecast volatility for an equity modeled using Black-Scholes is the standard deviation of the volatility time series multiplied by the square root of the number of intervals. For example, in an equity situation, if a yearly volatility is desired based on the log based volatility at the end of each week, there would be 52 intervals. Thus, the predicted volatility would be the standard deviation of the volatility series multiplied by the square root of 52. For the insurance premium setting, other choices for the volatility can also be used. For example, rather than using the standard deviation, the median value of the volatility time series can be used.

In various embodiments, the square root factor used for calculating the volatility does not need to strictly correlate with the number of intervals when using Black-Scholes to evaluate an insurance premium. For example, the number of days claims are received from a hospital could be each day of the year. Rather than correlating the square root factor with the number of intervals, it has been found that a smaller value is beneficial. Thus, instead of using the square root of 365 or the square root of 252 (number of normal business days in a year), a square root factor such as the square root of a number between about 15 and about 30 is more appropriate. In the examples provided herein, the square root of 20 was used as the square root factor for calculating a forecast volatility.

### Example of Determining a Premium—Black-Scholes

One alternative for determining a premium for the insurance product is to use a model for determining an equity option price, such as the Black-Scholes model. For example, the variables needed to apply a Black-Scholes model are:
S: the price of the underlying asset.
E: the strike price, equal to the deductible agreed with the insurer or reinsurer.
r: the interest rate. This is the rate standing at the time the option is valued.
t: the time to maturity. In general, the reinsurance is underwritten annually and the right to compensation can only be exercised at the end of that period. This approximation can also be used for a more conventional personal medical insurance product, even though claims may be paid throughout the course of the year.
σ: the volatility of the price of the underlying asset, as reflected by the claims accruing in the pool of insureds.

Once the values of these variables are determined, the premium can be calculated directly with the well-known Black-Scholes model given by the equations:

$$C = S * N ( {d}_{1} ) - E * N ( {d}_{2} ) * {e}^{- r t}$$

$${d}_{1} = \frac{[ ln ( \frac{S}{E} ) + ( r + {σ}^{2} / 2 ) t ]}{σ \sqrt{t}}$$

$${d}_{2} = {d}_{1} - σ \sqrt{t}$$

where N(⋅) is the cumulative normal distribution function, σ is the predicted or estimated volatility, and the underlying asset does not generate any return. As part of applying the Black-Scholes model, the following hypotheses can also be assumed. One assumption is that the underlying asset follows a continuous Gauss-Wiener stochastic process, so that the data is random on a local level. Second, it is assumed that volatility is the same for all persons insured. Another assumption can be that insurance coverage is purchased by buying a call (i.e., insurance) for each person that is insured by the insurer. For convenience, any insured persons whose annual hedging premium falls below a threshold level, such as less than $1, can be neglected. Finally, it can be assumed that the mean values and the distribution of the accumulated values are constant between consecutive periods.

One variable that is needed when using a Black-Scholes type model is the volatility (σ) of the yield of the underlying asset. As it is not an observable variable, a hypothesis must be assumed in order to estimate volatility from the information available at the moment of the valuation. To achieve this aim, the volatility is calculated based on the median value of the volatility time series multiplied by a square root factor (such as the square root of 20) as described above.

After determining the volatility, the other parameters of the model can be defined in order to calculate the premium. One parameter is the market price of the underlying asset. In an embodiment, the underlying asset price can be defined as the accumulated cost over the previous 12 months (or another insurance time period). However, insurance is typically priced based on coverage for a following period, rather than a preceding period. In order to estimate the asset price for a future 12 months, it can be assumed that the distribution of accumulated bills does not change between consecutive periods.

### Example of Determining Risk Price—Monte Carlo Simulation

The Black-Scholes equation provides a way for determining a value based on modeling claims data as points from a data distribution. By picking a known distribution with a convenient functional form, such as the normal distribution, a numerical solution to the equation is readily available. This allows for calculation of a value for the insurance premium. However, in some situations the historical data may not be suitable for modeling using an equation such as Black-Scholes. For example, one or more of the values in the Black-Scholes equation may have an associated uncertainty that needs to be incorporated into the premium price answer. Variations in one value can potentially be addressed by performing multiple calculations using the model. However, if more than value should be varied at a time and/or the variation in the value is not from a simple distribution, it may be difficult to manually determine the appropriate calculations to perform. Additionally or alternately, the distribution of claims may not be readily modeled by a mathematical distribution that can be easily worked with. For example, the Black-Scholes model expects data that corresponds to a Gaussian distribution, where the likelihood of a given claim value decreases as the claim value is farther away from some most probable claim value. While medical claims may appear to have this structure, it is not required. As a result, the actual distribution of claim values may result in a claim data set that is not suitable for use with the Black-Scholes calculation.

In situations where there are multiple uncertainties in the variables and/or the frequency distribution of the claims does not correspond to a distribution that is convenient for calculation, a Monte Carlo method can be used to predict average behavior. In a Monte Carlo method, an initial configuration for a system is specified. At each step, a proposed random change in one or more variables for the system is offered. If the change is accepted, the system is evaluated at the new configuration. This process can be repeated to generate a representative sampling of states for the system. Generating this representative sampling of states corresponds to sampling the distribution for the system. The sampled states can then be mathematically combined, such as by averaging, in order to obtain desired values regarding the system.

Because changes to the system are proposed within the simulation, any type of method can be used to generate the proposed changes. In particular, any convenient type of distribution can be used to generate changes for any variable of interest. As a result, a system having a distribution that is not easily evaluated mathematically can still be sampled effectively using a Monte Carlo method. Additionally, it is noted that a Monte Carlo method does not require any particular relation between the claim values at consecutive time intervals. Instead, the Monte Carlo method allows for direct sampling of the claim distribution in the frequency domain.

For example, consider a situation where the historical data for a particular type of claim shows a distribution where 80% of the claims are considered low value based on a dollar value of the claims, 1% of the claims are considered to be intermediate value, and 19% of the claims are considered high value. Additionally, the high value claims are distributed randomly throughout the time series, so that the volatility of the time series is large. Such a data set is not readily modeled using the normal distribution of the Black-Scholes equation. By contrast, a variety of choices are available for modeling this distribution in the frequency domain by a Monte Carlo technique. One possibility could be to use an explicit bi-modal distribution functional form to represent the data. Another option is to use a plurality of distribution functions, such as a plurality of Gaussian distributions centered on various claim values. The plurality of distribution functions could be fit to the historical data to determine weights for each of the Gaussians. The Monte Carlo technique could then sample from the various Gaussian distributions based in part on the determined weight for each Gaussian. By performing a plurality of Monte Carlo simulations with a sufficient sample size, statistics can be generated to determine an average expected claim value, as well as a standard deviation. A premium can then be selected based on a desired level, such as a premium sufficient to cover a claim value that is 2 standard deviations from the average.

### Example of Determining Risk Price—Jump Diffusion

As still another example, a jump-diffusion model can potentially be used to model a distribution of claims. In a jump diffusion model, a daily claim value can be modeled as a “particle” that moves (i.e., changes value) according to rules provided by the model. A jump-diffusion model includes two sources of motion. One source of motion or value change is motion generated by sampling from a distribution based on Brownian motion. This is the “diffusion” portion of the model. A second source of motion is occasional discontinuous “jumps” in position based on events generated from a Poisson distribution. The frequency of the discontinuous jumps can be varied to correspond, for example, to a frequency of occurrence of high claim volatility events. While some closed form solutions exist for particular formulations of a jump-diffusion model, Monte Carlo techniques can also be used for sampling the phase space of a jump-diffusion model.

In embodiments, selection of one of the above methods for determining a premium can optionally be based on fitting various types of distributions to a data set. An error in the fit of the distribution to the data can then be calculated. Based on the errors associated with various distribution fits, an appropriate model can be selected. For example, a hierarchy of model preferences can be established. If a normal distribution fits the data with an error below a first threshold value, the normal distribution can be selected for use regardless of the fit of the other distributions. In this situation, the Black-Scholes model can be used to determine a premium. If the fit of the normal distribution has an error above the first threshold, the fit based on other distributions can be considered. This can lead to selection of other models, such as the jump-diffusion model. Alternatively, this can lead to a determination that a Monte Carlo technique is more appropriate for evaluating the premium.

### Additional Verification

As an optional step, the premium generated using a Black-Scholes calculation (or another type of modeling) can be compared with the premium value determined by a conventional actuarial method, such as a Panjer Recursion calculation. One use for a Panjer Recursion calculation is to determine the minimum premium for avoiding insolvency of an insurance pool with varying levels of certainty. Performing a Panjer Recursion calculation on the original time series of claims data can thus be used as another way to verify that the historical claims have an appropriate distribution. For example, if the Black-Scholes calculation is used to determine a premium for a data set that cannot be represented well by a normal or Gaussian distribution, the Black-Scholes calculation may return a premium value that is too low. If an upper prediction limit determined by Panjer Recursion, such as the 95% upper prediction limit, is greater than the value determined by a Black-Scholes calculation, then the premium determined by Black-Scholes should likely be discarded.

### EXAMPLE 1

For an empirical application of the proposed valuation method, clinical and health claims records were randomly selected from a datamart extract from Cerner's Health Facts® data warehouse, which is a de-identified, HIPAA-compliant data warehouse derived from electronic health records (EHR). The datamart extract pertained to 22 U.S. acute-care institutions randomly selected so as to resemble an ‘integrated delivery network’ (IDN) of affiliated institutions comprising a typical U.S. health system. The dollar values in the datamart extract were normalized to 2010 dollars to allow comparison between different years. For this example, bills corresponding to the period between January 2007 and December 2010 that involved claims over $1,000,000 were selected. The analysis period was also divided into two sub-periods. A first sub-period was from January 2007-December 2008. This data was used as the “historical” data set for the example. The second sub-period was from January 2009-December 2010. In this example, the “historical” data is used to estimate a volatility for use in a Black-Scholes calculation for determining an insurance premium. This premium is then compared with the “future” claim values from the data in the second sub-period as a validation of the method.

As described above, one variable that needs to be determined to evaluate a premium using the Black-Scholes model is the volatility of the yield of the underlying asset. As it is not an observable variable, the volatility is estimated from the information available at the moment of the valuation. The volatility was determined by constructing a time series for the yield of the underlying asset and then estimating the volatility from this time series.

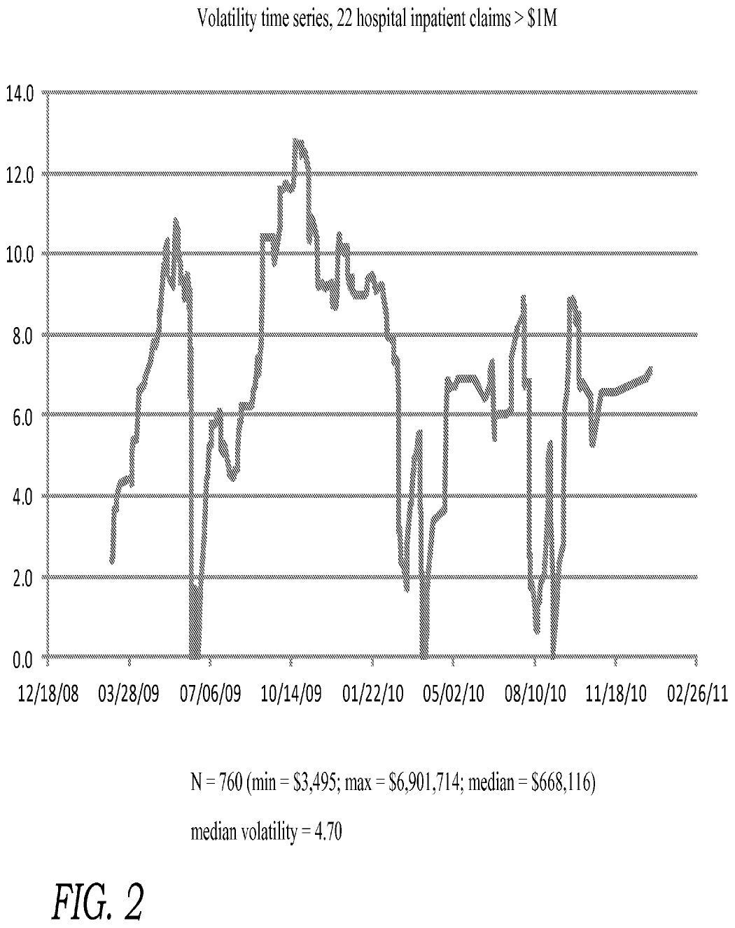

To construct the time series, the daily mean value of the medical bills recorded by the insurer was calculated for all the days in the calculation period (January 2007-December 2010). From this information, the accumulated value of daily claims for each day was calculated based on the claims from the previous 12 months. Next, the daily variation of these accumulated claim values was estimated in relative terms. A logarithmic approximation was used to obtain a one-year time series of the natural log of the daily returns of the underlying asset. From this, a historical volatility time series was determined. FIG. 2 shows the resulting historical volatility time series from the mining and processing of the data. The volatility of this log scale time series was used to estimate a predicted volatility for the Black-Scholes model calculation.

Prior to applying the Black-Scholes model, some additional tests were performed on the data from the two sub-periods in order to verify that the data did not contain any internal correlations that might influence the results. First, a test was performed to determine the possible existence of an ‘autoregressive conditionally heteroscedastic’ (ARCH) pattern. Both the autocorrelation and partial autocorrelation functions were studied for the full data set. The autocorrlation and partial autocorrelation functions did not suggest the existence of an autoregressive or moving average pattern for the daily variation of accumulated medical bills. This was further confirmed by the Ljung-Box test. As a result, it was determined that the claims data series used for this example did not contain a statistically significant autocorrelation.

After ruling out autocorrelation in the series of claims data, the existence of autocorrelation in the square of the variable was also tested. Once again, the Ljung-Box test indicated no autocorrelation and therefore suggested that an ARCH pattern is not probable. The Lagrange multiplier test confirmed rejection of an autoregressive conditionally heteroscedastic pattern at the p<0.05 level.

After determining that the claims data represented a data set without autocorrelation, the claims data was used to determine the volatility for the “historical” data in the first sub-period. This volatility was then used as a forecast the volatility for the second sub-period of January 2009-December 2010. This allowed for a comparison between the actual claim behavior in the second sub-period relative to the premium value determined based on the Black-Scholes model according to an embodiment of the invention. The volatility was estimated as the standard deviation for the daily return of accumulated claims during the period January 2009-December 2010. To determine this estimated volatility, the median volatility value for the period January 2007-December 2008 was calculated and used as a forecast for the volatility from January 2009-December 2010.

After determining the volatility, the other parameters of the model were defined in order to calculate the premium. The value of the underlying asset was defined as the value of the accumulated cost over the previous 12 months. Because an insurance premium is determined at the beginning of an insurance period, the underlying asset value needs to be estimated. Thus, the asset value was estimated based on the assumption that the distribution of accumulated bills does not change between consecutive periods.

This hypothesis of using the past distribution to model the future distribution was tested at the moment the premiums would have been valued (January 2009) by comparing the statistical distribution of the accumulated costs for two consecutive annual periods where data was available: January 2007-December 2008 and January 2009-December 2010. No significant differences were observed between the stop-loss claims patterns of the two periods. This was further supported by using the Kolmogorov-Smirnov test, where the null hypothesis is the equality between the distribution functions for both periods against the alternative hypothesis that assumes that the statistical distribution for the period between January 2007-December 2008 is different from that for the period January 2009-December 2010. The value for the Kolmogorov-Smirnov statistic was 0.064, while the critical value for a 99% confidence level was 0.068. Thus, both time periods appear to have the same statistical distribution at a 99% confidence level. In other words, the statistical distribution for the variable studied (the daily return of the accumulated stop-loss cost) was not changed between the two consecutive time periods. This indicated that prior data should be suitable for modeling a future value.

By using values calculated from the “historical” data sub-period, the minimum premium for 2010 for a $1,000,000 stop-loss policy for the 22 IDN-affiliated insureds was calculated based on accumulated stop-loss claims at 31 Dec. 2009 ($304,145,645) and a median volatility of 4.70. The minimum premium calculated by the Black-Scholes model for a call option (insurance product) with a strike price equal to the 95% prediction limit based on the 2009 claims was $566,453,623, or an average of $25,747,892 per IDN-member institution. The actual accumulated stop-loss claims during 2010 were $261,296,529 for this group of 22 institutions.

### EXAMPLE 2

In Example 1, the estimated premium value was inside the range estimated using actuarial techniques. However, the estimate described in Example 1 did not taken into account the existence of a $1,000,000,000 top, above which the IDN pays the excess. This variable is easily introduced into the model. As stated above, this involves the simple design of an exotic option contract that replicates this situation. This would be equivalent to assuming that, when the described call style option (strike price: $304,000,000, one-year period, etc.) is bought from the reinsurer, a call is simultaneously sold to the reinsurer with the same features, but at a strike price of $1,000,000,000. When both options are negotiated, the coverage situation can be described as follows: If the price of the underlying asset lies below $304,000,000, the buyer of the call (the IDN) does not exercise its right, but nevertheless pays the total accumulated value of the bill (plus the net premium, NP). The buyer of the call with a strike price of $1,000,000,000 would not exercise its right either. If the underlying price lies between $304,000,000 and $1,000,000,000, the reinsurer will not exercise the call it bought, but the IDN will exercise its option and pay the maximum value of $304,000,000 (plus the net premium). Finally, if the underlying price lies above $1,000,000,000, both parties will exercise their options; the reinsurer (as seller of a call with a strike price of $304,000,000) will pay the IDN the difference between the price of the underlying asset (C) and this strike price. But the IDN must also pay the reinsurer the difference between the price of the underlying asset and the strike price of the second of the options ($1,000,000,000). Thus, the net payment of the IDN would be the deductible plus the excess of the cost of the claims on the top, plus the net premium: C+NP+304,000,000−1,000,000,000=304,000,000+(C−1,000,000,000)+NP=C+NP−400,000,000.

For the IDN or reinsurer, selling a call option represents an income or a lower premium to be paid to the reinsurer for the coverage. Therefore, with regard to the call options bought by the insurer, we calculated the amount corresponding to the premiums of the calls sold by this firm. The data used are the same, with the exception of the strike price, which is now $1,000,000,000. As the top was set at such a high level, with respect to the normal accumulated annual bill, almost all the premiums are lower than $1. We only obtain premiums above $1 for values of the underlying asset above $115.000.000, although the amount is still insignificant. As a consequence, the existence of this top boundary has no significant impact on the valuation of the premiums to be paid by the IDN or the reinsurer.

### EXAMPLE 3

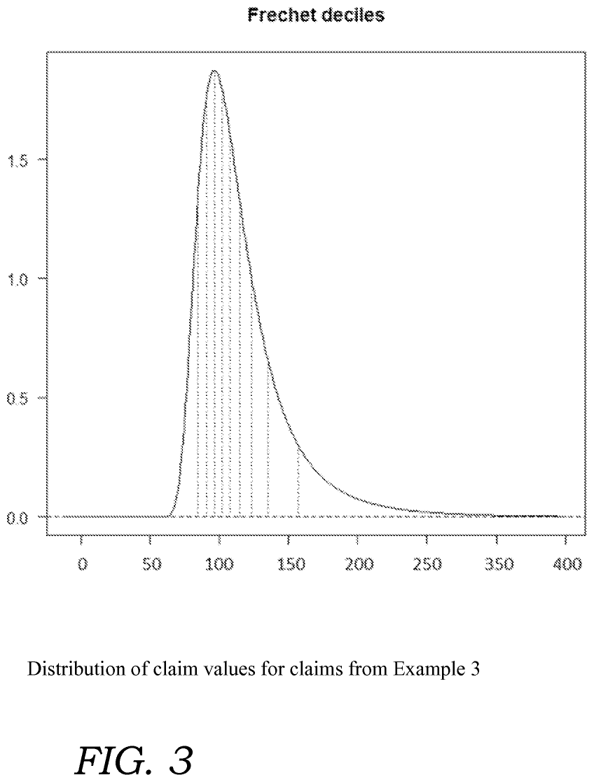

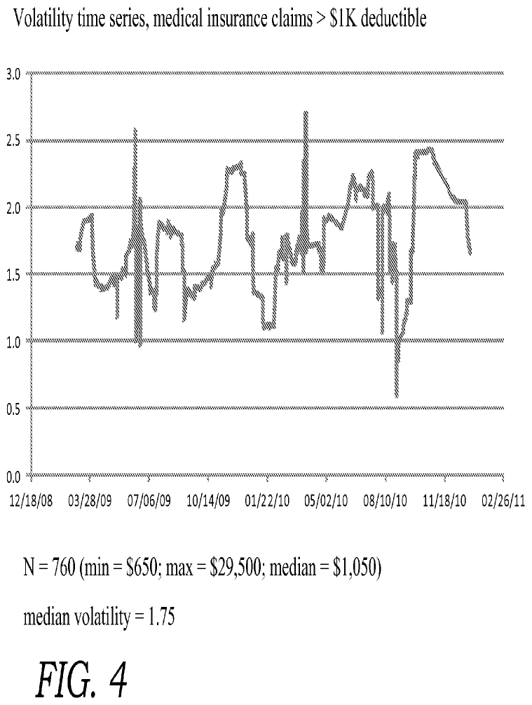

For a third empirical application of the proposed valuation method, clinical and health claims records were randomly selected from a datamart extract for individual health plan members and their beneficiaries. The claims were Fréchet-distributed, as shown in FIG. 3. The minimum premium for 2010 for a $1,000 deductible policy for the insureds was estimated, considering their accumulated stop-loss claims at 31 Dec. 2009 ($4,304,800) and a median volatility of 1.75. The volatility time series data for determining this premium is shown in FIG. 4. The minimum premium calculated by the Black-Scholes model for a European-style call option with a strike price at the 95% prediction limit for the 2009 claims was $4,903,055. The actual accumulated claims during 2010 was $4,423,400 for this group of insureds.

### Example of Operating Environment

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

As one skilled in the art will appreciate, embodiments of our invention may be embodied as, among other things: a method, system, or set of instructions embodied on one or more computer readable media. Accordingly, the embodiments may take the form of a hardware embodiment, a software embodiment, or an embodiment combining software and hardware. In one embodiment, the invention takes the form of a computer-program product that includes computer-usable instructions embodied on one or more computer readable media.

Computer-readable media include both volatile and nonvolatile media, removable and nonremovable media, and contemplates media readable by a database, a switch, and various other network devices. By way of example, and not limitation, computer-readable media comprise media implemented in any method or technology for storing information. Examples of stored information include computer-useable instructions, data structures, program modules, and other data representations. Media examples include, but are not limited to information-delivery media, RAM, ROM, EEPROM, flash memory or other memory technology, CD-ROM, digital versatile discs (DVD), holographic media or other optical disc storage, magnetic cassettes, magnetic tape, magnetic disk storage, and other magnetic storage devices. These technologies can store data momentarily, temporarily, or permanently. In an embodiment, the computer-readable media are tangible computer-readable media. In an embodiment, the computer-readable media are non-transitory computer-readable media.

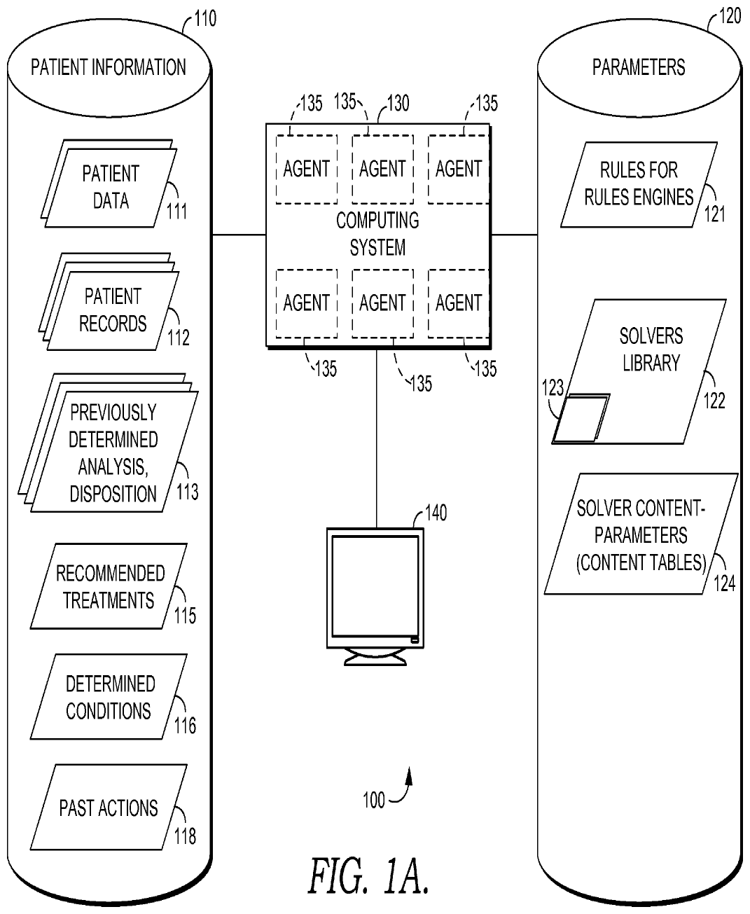

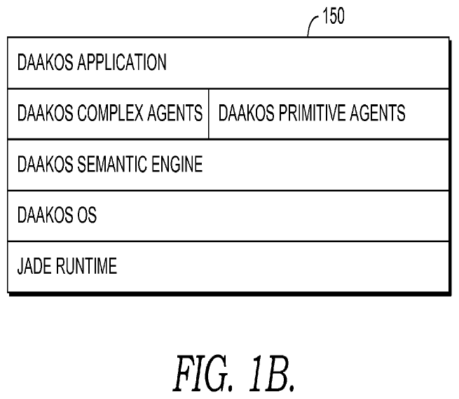

Referring to the drawings in general, and initially to FIG. 1A in particular, an exemplary operating environment 100 is provided suitable for practicing an embodiment of our invention. We show certain items in block-diagram form more for being able to reference something consistent with the nature of a patent than to imply that a certain component is or is not part of a certain device. Similarly, although some items are depicted in the singular form, plural items are contemplated as well (e.g., what is shown as one data store might really be multiple data-stores distributed across multiple locations). But showing every variation of each item might obscure the invention. Thus for readability, we show and reference items in the singular (while fully contemplating, where applicable, the plural).

As shown in FIG. 1A, environment 100 includes computer system 130. In some embodiments, computing system 130 is a multi-agent computing system with one or more agents 135, as shown in FIG. 1A and described in greater detail below. But it will be appreciated that computing system 130 may also take the form of a single agent system or a non-agent system. Computing system 130 may be a distributed computing system, a centralized computing system, a single computer such as a desktop or laptop computer or a networked computing system.

In some embodiments of our invention, computer system 130 is a multi-agent computer system with agents 135. Multi-agent system 130 may be used to address the issues of distributed intelligence and interaction by providing the capability to design and implement complex applications using formal modeling to solve complex problems and divide and conquer these problem spaces. Whereas object-oriented systems comprise objects communicating with other objects using procedural messaging, agent-oriented systems use agents 135 based on beliefs, capabilities and choices that communicate via declarative messaging and use abstractions to allow for future adaptations and flexibility. An agent 135 has its own thread of control which promotes the concept of autonomy.

Embodiments using multi-agent system 130 provide capabilities to adapt the frequency and messages used for communication between the system 130 and one or more users 140, based on changes to the environment and provide capabilities to filter out noisy data, thereby providing more flexible and adaptable decision making abilities. In some embodiments, this is accomplished by using leveraging preceptors and effectors. Preceptors or sensors, which in some embodiments may be agents, detect changes in an operating environment and pass this information to the agent system. Effectors, which in some embodiments may be agents 135, respond directly to changes in an operating environment and consider goals and alternatives prior to implementing a change to the environment.

Embodiments using multi-agent system 130 further have the capability of supporting intelligent information retrieval and filter out noisy data and utilize heuristics to narrow down a search space to assist in solving complex problems. The multi-agent system 130 facilitates designing individual agent behaviors and their interactions with other agents 135 and with users 140. In some embodiments, agents 135 encoded with both declarative and procedural knowledge and can therefore learn by means of exploration of knowledge and imitation of other agents, for example, by leveraging aggregation of bottom-up and top-down modeling. In some embodiments, the agent system 130 accepts an abstract workflow and converts it into an actual executable workflow, by for example, using contract and negotiation in multi-agent system 130. The executable workflow may then leverage agents to run the actual workflow.

Embodiments using multi-agent system 130 coordinate the actions of the agents 135 to cooperate to achieve common objectives, and negotiate to resolve conflicts, which allows for adaptability, flexibility, and organizational relationships. The transformation of heterogeneous knowledge and content into homogeneous knowledge and content is an important trait of the multi-agent system to provide interoperability. The multi-agent system 130 operates to achieve its goals while still interacting with agents, including agents outside of the multi-agent system 130 (not shown) and users 140 at a higher degree of flexibility. As an example, in one embodiment a multi-agent system 130 can be utilized to efficiently determine a method for calculating a premium value. An agent can receive input defining the type of insured pool to be considered. The available historical data is mined to extract a relevant historical data set. Optionally, the data set can then be analyzed to determine an appropriate distribution for modeling the historical data set. Based on the distribution, an equation-based solution such as Black-Scholes or jump-diffusion can be selected, or Monte Carlo sampling of the distribution can be used to generate a premium value.

In some embodiments, agents 135 continually monitor events to proactively detect problems and leverage reasoning to react and dynamically alter a plan. Practical reasoning involves managing conflict resolution where the relevant considerations are provided by the agent's desires about what the agent believes. This involves deliberation by deciding what state of affairs the agent wants to achieve using intentions and by means-end reasoning which is how to achieve those desires using plans. By way of background, an intention is stronger than a desire and planning achieves designated goals. Thus in one embodiment, a basic planning module consists of goals and intentions to be achieved, actions that can be performed, and a representation of the environment. These plans can thus handle priorities, uncertainty and rewards to influence the actual plans. An agent has its own thread of control which promotes the concept of autonomy. Additional information about the capabilities and functionality of agents and distributed multi-agent operating systems, as they relate to our invention, is provided in U.S. patent application Ser. No. 13/250,072, filed on Sep. 30, 2011, which is herein incorporated by reference in its entirety.

Continuing with FIG. 1A, system 130 is communicatively coupled to patient information 110 and parameters 120, and user interface 140, described below. System 130 performs processing on claims information 110 and parameters 120. In some embodiments, system 130 includes one or more agents 135, which process patient information 110 using parameters 120 to determine historical data sets corresponding to a potential insured group and/or potential insured product, to identify distributions with suitable fits to a historical data set, to determine a premium value based on a model or simulation, or to invoke other agents, such as agent solvers, to perform these determinations.

System 130 is executed by or resides on one or more processors operable to receive instructions and process them accordingly, and may be embodied as a single computing device or multiple computing devices communicatively coupled to each other. In one embodiment processing actions performed by system 130 are distributed among multiple locations such as a local client and one or more remote servers. In another embodiment, system 130 resides on a computer, such as a desktop computer, laptop, or tablet computer. Example embodiments of system 130 reside on a desktop computer, a cloud-computer or distributed computing architecture, a portable computing device such as a laptop, tablet, ultra-mobile P.C., or a mobile phone.

Coupled to system 130 is display for user 140. Display for a user 140 provides a presentation capability and user interface to facilitate communication with users. Using display for a user 140, a user may view determined results about a patient or provide additional information such as patient information, in one embodiment. Display for a user 140 may be a single device or a combination of devices and may be stationary or mobile. In some embodiments, a user interface on display device takes the forms of one or more presentation components such as a monitor, computing screen, projection device, or other hardware for displaying output. In some embodiments, a user interface on display device takes the form of one or more presentation components with user input components, such as a remote computer, a desktop computer, laptop, pda, mobile phone, ultra-mobile pc, computerized physician's clipboard, or similar device. In some embodiments, data elements and other information may be received from display device by a user 140. Queries may be performed by users 140; additional orders, tests, feedback or other information may be provided through the display device to user 140.

Environment 100 includes data store 110 which includes claims information and data store 120 which includes parameters. In some embodiments, data stores 110 and 120 comprise networked storage or distributed storage including storage on servers located in the cloud. Thus, it is contemplated that for some embodiments, the information stored in data stores 110 or 120 is not stored in the same physical location. For example, in one embodiment, one part of data store 110 includes one or more USB thumb drives or similar portable data storage media. Additionally, information stored in data store 110 and 120 can be searched, queried, analyzed using system 130 or user interface 140, which is further described below.

Data store 110 can include claims data from a variety of sources. Examples of sources can include claims data from traditional hospitals, walk-in clinics, urgent care facilities, and other locations that render medical services. Claims data can also be retrieved from centralized data sources such as health information exchanges. Claims data from any other convenient source can also be included.

Data store 120 comprises parameters and information associated with the multi-agent system 130. Although depicted as working with a multi-agent system, in one embodiment, data store 120 works with single-agent system parameters and information, or non-agent system parameters and information. In one embodiment, data store 120 includes rules for a rules engine 121, and solvers library 122. Rules for a rules engine 121 include a set of rules or library of rules. In one embodiment, rules 121 are usable by an expert rules-engine, such as an agent 135 in multi-agent system 130. Alternatively, in non-agent embodiment, rules 121 include a library of rules usable by non-agent processes. One example application of rules 121 by a rules engine includes determining actions or dispositions associated with a patient, from a number of determined conditions or recommended treatments.

Solvers library 122 includes one or more solvers, which can include non-agent solvers, agent solvers (discussed below) or both. In some embodiments, solvers, which may also be referred to as “resolvers,” are applied to determine one or more conditions or recommended treatments for a patient. A finite state machine solver may be used to determine the conditions and recommended treatments of a patient suffering from a number of conditions including congestive heart failure. Solvers may also invoke or apply other solvers. Continuing this example, the finite state machine agent solver may invoke a linear solver, such as a mixed integer linear solver, to evaluate each state in order to determine the patient's condition. In one embodiment, the finite state machine returns the actual state for each clinical condition of the patient, which is then passed on to the mixed integer linear solver as parameters, to apply the mixed integer solver based on the clinical state, and content tables 124. The solvers library 122 can be updated as new solvers are available. Another example solver is the data-extraction solver, which is described in further detail below. A data-extraction solver is a type of solver that is applied to unprocessed patient information, such as a physician's narrative or patient results data, in order to generate discretized data that is usable for other solvers.

In some embodiments, agents 135, facilitate solving problems including the problems described above, by employing one or more solvers, from library of solvers 122. Furthermore, where existing rule systems may utilize forward chaining, backward chaining and combination, agents 135 can integrate these rule capabilities as well as other traditional and heuristic techniques. These agents 135, which may be referred to as agent solvers, can also leverage the best techniques for the problem at hand. They can register their abilities to the overall system and coordinate and communicate with other agents, users, or the overall system, to solve problems based on their current capabilities. Still further, new or improved solvers, which may be introduced at future times, are able to be leveraged by swapping out current agents with new agents dynamically and without the need to recompile or reconfigure the system. Thus embodiments using multi-agent system 130 can provide advantages, in some scenarios, over single-agent systems and non-agent systems. By analogy, a single celled organism is analogous to a single-agent system, while a complex multi-celled organism is analogous to the multi-agent system. Accordingly, the “reasoning” capabilities of multi-agent system 130 are superior to the “reasoning” exhibited by a single-agent system, and the multi-agent system 130 is not constrained at design time and has the ability to grow and adapt over time based on future needs not anticipated at the time of instantiation or design.

In some embodiments, agents 135 provide enhanced decision support by using multi-agent properties like collaboration, persistence, mobility and distributed-operation, autonomy, adaptability, knowledge and intelligence, reactive and proactive capability, reuse, scalability, reliability, maintainability, security, fault tolerance, trust, and other primary properties. In addition, numerous secondary properties of multi-agents in embodiments of our invention may facilitate decision support, including: reasoning, planning and learning capabilities; decentralization; conflict resolution; distributed problem solving; divide-and-conquer strategies for handling complex problems; location transparency; allowing for competing objects to be represented; goal-driven or data driven including agent to agent or user to agent; time driven; support for multiple layers of abstraction above services thereby providing flexibility, adaptability, and reuse and simplification; negotiation; hierarchies having dynamic self-organization; abilities to spawn and destroy agents as needed; utilization of transient and persistent data; abilities to address uncertain, missing or inconsistent data; sensitivity to resource and time constraints; ontology-driven functionality; flexible run-time invocation and planning; obligations; ability to act to achieve objectives on behalf of individuals and organizations; organizational influence; and other secondary properties. Examples of agents, which may be used by the multi-agent systems of embodiments of our technologies, include: Interface agents; planning agents; information agents; adapter wrapper agents; filter agents; discovery agents; task agents; blackboard agents; learning agents, including supervised learning, unsupervised learning, reinforcement learning, for example; observer agents; inference agents; communication agents; directory agents; administrator and security agents; facilitator agents; mediator agents; and agent solvers. Agent solvers can include, for example: markov decision processing; approximate linear programming; natural language extraction solvers (e.g., nCode); fuzzy-neural networks, logistic and linear regression; forward chaining inference (e.g., data driven); backward chaining inference (e.g., goal driven); inductive inference; genetic algorithm; neural network including with genetic algorithm for training; stochastic; self-organizing Kohenen map; Q-learning; quasi-Newton; gradient; decision trees; lower/higher bound search; constrain satisfaction; naives bayes fuzzy; LP-solver including mixed integer multi-variable min/max solvers; Finite State Machine and HFSM; temporal difference reasoning; data mining for classification, clustering, learning and prediction; K-means; support vector machines; K-nearest neighbor classification; C5.0; apriori; EM, simulated annealing, Tabu search, multi-criteria decision making, evolutionary algorithm, and other similar solvers.

In some embodiments, where particular types of agent solvers are more efficient at handling certain patient scenarios, a planning agent may invoke the particular type of agent solver most appropriate for the scenario. For example, a finite state machine agent solver and a liner solver agent solver may be invoked by a planning agent, in a scenario involving a patient experiencing congestive heart failure.

Continuing with FIG. 1A, some embodiments of multi-agent system 130 employ decision making for applications including, for example, searching, logical inference, pattern matching and decomposition. A subset of solvers library 122 includes decision-processing solvers 123. Decision processing solvers 123 are a special set of solvers used for decision making, although it is contemplated that in some embodiments any solvers of solvers library 122 or solver agent may be used for decision processing. Examples of agent decision procession applications include: searching, including heuristic and traditional searching; list; constraint satisfaction; heuristic informed; hill climbing; decision tree; simulated annealing; graph search; A* search; genetic algorithm; evolutionary algorithm; tabu search; logical inference; fuzzy logic; forward and backward chaining rules; multi-criteria decision making; procedural; inductive inference; pattern recognition; neural fuzzy network; speech recognition; natural language processing; decomposition; divide and conquer; goal tree and sub-goal tree; state machine; function decomposition; pattern decomposition; and other decision processing applications. In some embodiments, agents designed or instantiated with a particular decision processing application may be swapped out, in a more seamless and transparent manner than with non-agent systems, with another agent having more advanced decision processing functionality as this becomes available or is needed.

Turning to FIG. 1B, an illustrative example is provided of a framework suitable for implementing a multi-agent system, such as computer system 130 of FIG. 1A, and is referenced generally by the number 150. Framework 150 has a layered architecture. At the lowest level depicted in the embodiment shown in FIG. 1B, framework 150 includes a layer for JADE runtime. In other embodiments, frameworks such as Cougaaar, Zeus, FIPA-OS, or an open-agent architecture, may be used. Although not a requirement, it is preferable that the framework include the following properties, which are present in the JADE framework: FIPA compliance; support for autonomous and proactive agents and loosely coupled agents; peer-to-peer communication; fully distributed architecture; efficient transportation of asynchronous messages; support for white and yellow page directory services; agent life-cycle management; agent mobility; subscription mechanism for agents; graphical tools for debugging and maintenance; support for ontology and content languages; library for interaction protocol; extensible kernel for extensions to build customized framework; in-process interface for launching and control; support for running agents on wireless mobile devices; integration with various web-based technologies; and pure Java implementation.

JADE, which is an acronym for Java Agent Development Framework is a middleware software development framework that is used for facilitating implementation of multi-agent systems. Specifically, the JADE platform includes functionality which facilitates the coordination of multiple agents, and functionality for facilitating the distribution of agent platforms across multiple machines, including machines running different operating systems. Moreover, JADE further includes functionality for changing system configuration at run-time by moving agents from one machine to another, as required.

Continuing with FIG. 1B, on top of the JADE runtime framework is the Distributed Adaptive Agent Knowledge operating system (“DAAKOS”). DAAKOS is a decision-support framework built upon JADE or another multi-agent framework. DAAKOS is a multi-agent framework with heuristic, adaptive, self-optimizing and learning capabilities and the ability to decompose complex problems into manageable tasks to assist clinical decision making at point of care. For example, care givers and other users can leverage this intelligent agent system to detect a change in personal health or to leverage up to date knowledge about medical conditions, preventive care, and other relevant interests. Accordingly, in one embodiment DAAKOS can be thought of as an intelligent, self-learning agent system using a cloud metaphor.

Specifically, DAAKOS utilizes multi-agents 135 that collaborate with each other and interface with external systems, services and users and has the ability to monitor changes and incorporate past knowledge into decision making in order to generate and evaluate alternative plans or adapt plans to handle conflict resolution and critical constraints. A multi-agent virtual operating system provides efficient means to build complex systems composed of autonomous agents with the ability to be reactive, persistent, autonomous, adaptable, mobile, goal-oriented, context aware, cooperative and able to communicate with other agents and non-agents. In some embodiments, intelligence is achieved within agents by way of support provided by a rich ontology within a semantic network. For example, a multi-level of collaborating agents 135 allows low level agents to transform data so that it can be passed on to another agent, and to continue the data transformation until the data has been completely transformed from bits of data which may sometimes represent incomplete, outdated, or uncertain data, to a form a usable collection of data with rich meaning. In this example, when it becomes necessary to attack complex problems the agent 135 is permitted to constrain and narrow its focus at an individual level to support decomposition. Domain specific agents can be leveraged in some embodiments to use an ontology to manage local domain specific resources.

The DAAKOS operating system layer handles process management, scheduling, memory, resource management, Input/Output (“I/O”), security, planning, as well as other processes traditionally handled by operating systems, and in some embodiments includes memory, which may include short, intermediate, and/or long term memory, I/O, internal agent blackboard, context switching, kernel, swapper, device resource management, system services, pager, process managers, and logging, auditing, and debugging processes. In some embodiments, the DAAKOS operating system layer is a distributed virtual operating system. On top of the DAAKOS operating system layer, in the embodiment illustratively provided in FIG. 1B, is the DAAKOS Symantec Engine, which provides the platform for DAAKOS agents 135. DAAKOS agents 135 include complex agents and primitive agents. On top of the agents layers are DAAKOS Applications. These include, for example, DAAKOS agent solvers such as a finite state machine instantiated and executed to determine a patient's conditions and recommended treatments, transactions knowledge engines, and other applications leveraging DAAKOS agents 135.

### Additional Embodiments

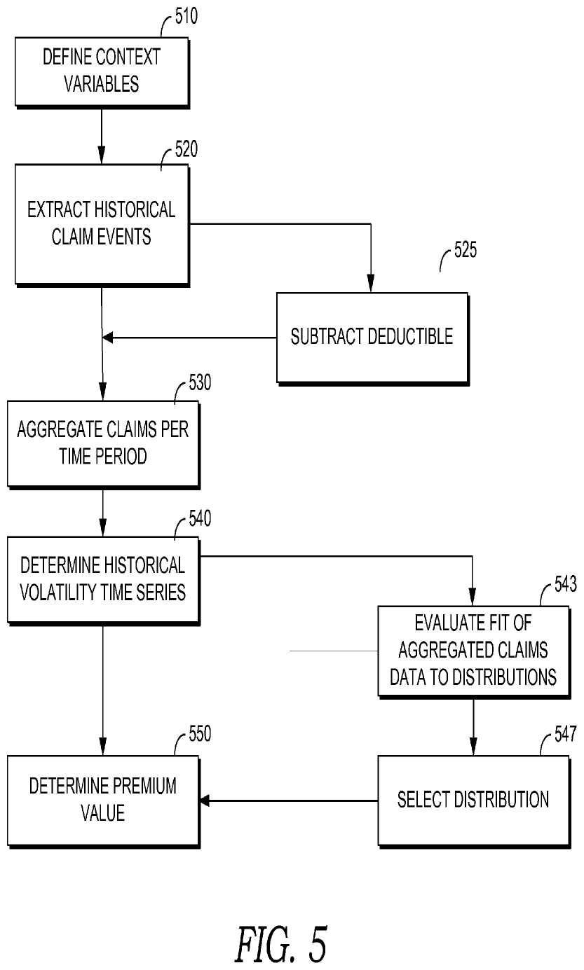

FIG. 5 shows an example of a process flow or method according to an embodiment of the invention that is suitable to be carried out using a multi-agent distributed computing system or another computing system, as described above. In FIG. 5, a process flow begins by defining 510 a plurality of context variables. The context variables defined in a particular embodiment may be dependent on the type of equation or modeling that will be used for determining a premium. Context variables can include the desired coverage amount, the term of the insurance, the deductible and/or the cap or stop-loss top, the statistical prediction limit (such as the number of standard deviations above the mean), the risk-free interest rate, the cost-of-carry, or other variables. Defining 510 of context variables can also include providing a definition of the group and/or conditions being insured, if the group being insured does not correspond to a random cross-section of all potential insureds. For example, an insurance or reinsurance policy might be specifically tailored to cover only heart-related diseases. In this situation, the only insurance events of interest are heart-disease related events. In an embodiment, the determination of which context variables to use is facilitated by an agent 135. In an embodiment, at least one context variable is provided by a user. In an embodiment, context variables are provided as goals or intentions to be achieved. In one embodiment, system 130 determines a type of modeling to be used for determining a premium, and defines context variables using information stored in patient information 110 and parameters 120, or by interrogating the user.

After providing a definition 510 of the context, historical events and corresponding payouts (claims) that match the context are extracted 520 from one or more sources of claims data. Depending on the embodiment, any deductible associated with the context variable can be subtracted from matching claims events during extraction 520, or subtraction of a deductible can be a separate process 525. The claims events can then be aggregated 530 based on a desired time period to form aggregated claims data per time period. For example, the aggregation can be used to form daily aggregated claims, weekly aggregated claims, or claims aggregated relative to any other convenient period of time. In one embodiment, the claims are aggregated in near-real time as claims data becomes available. The aggregated claims data per time period can then be used to determine 540 into a historical volatility time series. Determining 540 a historical volatility time series can include converting the aggregated claims data per time period into a time series of differences between logarithms of the aggregated claims data. After converting the aggregated claims data into a historical volatility time series, at a step 543 the data can optionally be evaluated for fit relative to one or more distributions. In an embodiment, an agent 135 is used to facilitate this evaluation. If, for example, it is known in advance that a particular calculation method will be used, such as Black-Scholes, the evaluation 543 of fit can be omitted. If the evaluation 543 of fit is used, a distribution suitable for use can be selected, at a step 547, based on the fit evaluation. A premium value is then determined, at a step 550, either using a predetermined calculation method or based on a method that was selected 547 based on the fit evaluation. In an embodiment an agent 135 facilitates the determination of a premium.

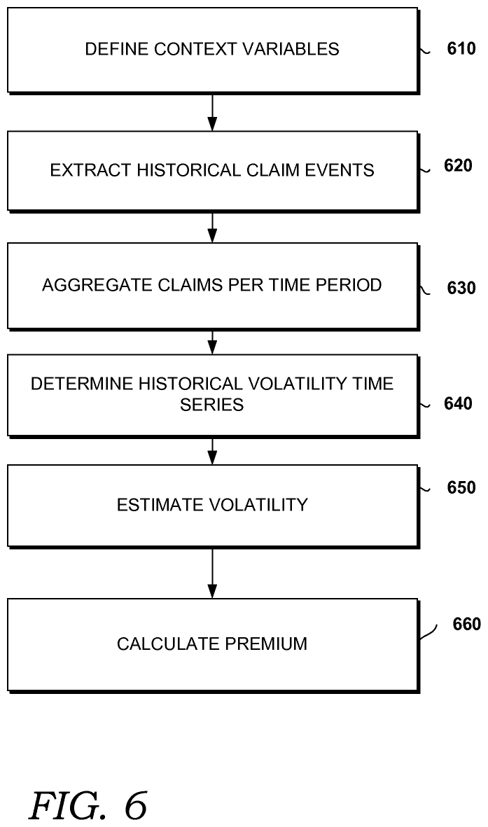

FIG. 6 shows another example of a process flow according to an embodiment of the invention. The process flow in FIG. 6 shows an example where the Black-Scholes method is used for determining a premium. Note that the example in FIG. 6 is compatible with FIG. 5, so the use of Black-Scholes can either be predetermined or based on a selection of Black-Scholes after verifying that a normal distribution provides a suitable fit for the historical claims data. As in FIG. 5, one or more agents 135 may be used to facilitate carrying the process shown in FIG. 6. In FIG. 6, context variables are defined 610. Based on the definition 610 of the context, historical claim events are extracted 620 that match the defined context. Any deductible can be removed from the extracted claims during extraction 620, or a separate process step (not shown) can be used. The extracted claim events can then be aggregated 630 based on desired time period, such as a daily time period. A historical volatility time series is then determined 640 for a plurality of time periods based on differences between logarithms of the aggregated claims data per time period. Based on historical volatility time series, a volatility is estimated 650. The volatility is then used to calculate 660 a premium value for the insurance, such as by using the Black-Scholes model.

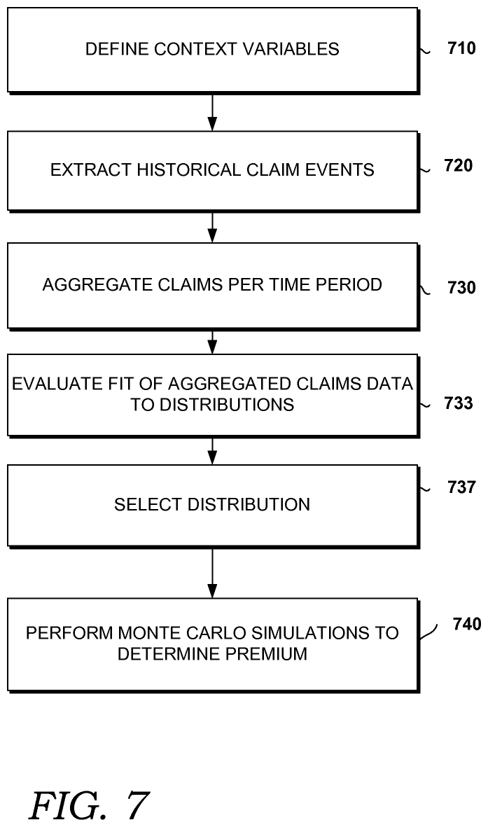

FIG. 7 shows still another example of a process flow according to an embodiment of the invention. In the embodiment shown in FIG. 7, a test of distribution fit to the data results in selection of a distribution that is evaluated using a Monte Carlo technique to sample the distribution. In FIG. 7, context variables are defined 710. Based on the definition 710 of the context, historical claim events are extracted 720 that match the defined context. Any deductible can be removed from the extracted claims during extraction 720, or a separate process step (not shown) can be used. The extracted claim events can then be aggregated 730 based on desired time period, such as a daily time period. One or more distributions are then evaluated 733 for fit to the aggregated claim events, and a distribution is selected 737 based on the evaluation. One or more Monte Carlo simulations are then performed 740 to sample the selected distribution in order to determine a premium.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

Accordingly, in one embodiment, a method is provided for determining a premium for insurance coverage. The method comprises defining a plurality of context variables related to the insurance coverage, at least one of the context variables corresponding to a feature of the insurance coverage. The method further comprises extracting, using a plurality of autonomous software agents, historical claim events from one or more sources of claims data based on the plurality of context variables. The method further comprises aggregating the extracted historical claim events based on a time period to form aggregated claims data per time period; determining a historical volatility time series based on differences between logarithms of the aggregated claims data per time period for a plurality of time periods; determining a fit of the logarithms of the aggregated claims data per time period relative to one or more distributions; selecting a distribution based on the determined fit relative to the one or more distributions; and calculating a premium for the insurance coverage based on the selected distribution.

In any of the above embodiments, the method further comprises determining a fit of the logarithms of the aggregated claims data per time period comprises determining a fit relative to a normal distribution, the normal distribution being selected if a fit parameter is less than a threshold value, and wherein the premium for the insurance coverage is calculated based on a Black-Scholes model.

In any of the above embodiments, the method further comprises determining a fit of the logarithms of the aggregated claims data per time period comprises estimating a volatility based on the historical volatility time series, wherein the normal distribution is selected if the estimated volatility is less than a threshold volatility value.

In any of the above embodiments, the method further comprises selecting a distribution based on the determined fit comprises: determining that a fit parameter for a normal distribution is greater than a threshold value; and selecting a distribution suitable for calculation of the premium using a jump-diffusion model.

In any of the above embodiments, the method further comprises calculating a premium for the insurance coverage based on the selected distribution comprises performing a plurality of Monte Carlo simulations using the selected distribution.

In any of the above embodiments, the selected distribution comprises a plurality of normal distributions centered at different values, each of the normal distributions having a sampling weight.

In any of the above embodiments, the method further comprises extracting historical claim events comprises filtering claim events to select claim events that match a claim value range specified by one or more defined context variables.

In any of the above embodiments, the historical claim events are extracted from a plurality of claim event sources, the plurality of autonomous agents extracting events from the claim event sources in parallel on a plurality of processors.

In one embodiment, one or more computer-readable media are provided having computer-executable instructions embodied thereon that when executed by a processor, facilitate a method for determining a premium for insurance coverage. The method comprises defining a plurality of context variables related to the insurance coverage, at least one of the context variables corresponding to a feature of the insurance coverage; extracting, using a plurality of autonomous software agents, historical claim events from one or more sources of claims data based on the plurality of context variables; aggregating the extracted historical claim events based on a time period to form aggregated claims data per time period; determining a fit of the logarithms of the aggregated claims data per time period relative to one or more distributions; selecting a distribution based on the determined fit relative to the one or more distributions; and performing a plurality of Monte Carlo simulations based on the selected distribution to determine a premium for the insurance coverage.

It will be understood that certain features and subcombinations are of utility and may be employed without reference to other features and subcombinations and are contemplated within the scope of the claims. Not all steps listed in the various figures need be carried out in the specific order described. Accordingly, the scope of the invention is intended to be limited only by the following claims.

What is claimed is:

1. A computer-performed method comprising:
defining a plurality of context variables related to an insurance coverage, at least one of the context variables corresponding to a feature of the insurance coverage;
extracting, using a plurality of autonomous software agents, historical claim events from one or more sources of claims data based on the plurality of context variables;
aggregating the extracted historical claim events based on a time period to form aggregated claims data per time period;
determining a historical volatility time series based on differences between logarithms of the aggregated claims data per time period for a plurality of time periods;
determining a fit of the logarithms of the aggregated claims data per time period relative to one or more distributions;
selecting a distribution based on the determined fit relative to the one or more distributions;
calculating a premium for the insurance coverage based on the selected distribution; and
causing to present, via a user interface, an indication of the premium.
